(19) United States
(12) Patent Application Publication
(10) Pub. No.: US 2008/0207490 A1
(43) Pub. Date: Aug. 28, 2008

(54) Glucose Uptake Modulator and Method for Treating Diabetes or Diabetic Complications

(75) Inventors: Kyung-Moo Yea, Kyungsangbuk-do (KR); Jae-Yoon Kim, Kyungsangbuk-do (KR); Jong-Hyun Kim, Kyungsangbuk-do (KR); Byoung-Dae Lee, Kyungsangbuk-do (KR); Seung-Je Lee, Kyungsangbuk-do (KR); Tae-Hoon Lee, Kyungsangbuk-do (KR); Pann-Ghill Suh, Kyungsangbuk-do (KR); Sung-Ho Ryu, Kyungsangbuk-do (KR)

Correspondence Address: LEXYOUME IP GROUP, LLC, 1233 TWENTIETH STREET, N.W., SUITE 701, WASHINGTON, DC, 20036, US

(73) Assignees: POSTECH Foundation, Pohang (KR); POSCO, Pohang (KR)

(21) Appl. No.: 11/994,380

(22) PCT Filed: Jul. 7, 2006
(86) PCT No.: PCT/KR06/02656

§ 371 Date: Dec. 31, 2007

Related U.S. Application Data
(60) Provisional application No. 60/595,457, filed on Jul. 7, 2005.

(51) Int. Cl. A61K 38/28; C07F 9/02; C07K 14/62; A61K 31/685; A61P 3/04; C07K 14/00; A61K 38/16

(52) U.S. Cl. 514/3; 558/170; 530/324; 530/303; 514/77; 514/12

(57) ABSTRACT

The present invention relates to an glucose uptake modulator, a pharmaceutical composition comprising the glucose uptake modulator, and a method of treating a diabetes or diabetic complications in a mammal in need thereof, which comprises administering to said mammal an effecting amount of a glucose uptake modulator.

### CROSS-REFERENCE TO RELATED APPLICATION

This application claims priority to and the benefit of U.S. provisional patent application No. 60/595,457 filed in the United State of America Patent & Trademark Office on Jul. 7, 2005, the entire contents of which are incorporated herein by reference.

### BACKGROUND OF THE INVENTION

(a) Field of the Invention

The present invention relates to a glucose uptake modulator, a pharmaceutical composition comprising the glucose uptake modulator, and a method of treating a diabetes or diabetic complications in a mammal in need thereof, which comprises administering to said mammal an effecting amount of the glucose uptake modulator.

(b) Description of the Related Art

In addition to insulin, various hormones or physiological conditions are capable of stimulating the glucose uptake. For example, exercise induces glucose uptake in skeletal muscle through an insulin independent pathway. Also, activation of α₁-adrenergic or endothelin_{A} receptors result in enhanced glucose uptake rates independent of insulin. Some of the signaling mechanisms that mediate these metabolic responses are similar to those utilized by insulin, whereas others are clearly distinct. For instance, the stimulation of glucose uptake that occurs in adipocytes treated with arachidonic acid, peroxisome proliferators activated receptor γ agonists seems to involve specific and insulin-independent signaling pathways.

For many years adipose tissue was viewed as playing a key role in total body lipid and energy homeostasis. Removal of excess glucose from the circulation involves the stimulation of glucose transport into adipose and muscle tissue. It has become clear that glucose intolerance in type 2 diabetes is manifested by defects in glucose transport into adipose tissue. Therefore, the finding of new endogenous factors which regulate glucose transport in adipocytes is essential for our understanding of diabetes process and for the development of improved therapeutic strategies.

Bioactive molecules such as hormones, neurotransmitters, and cytokines play important roles in many regulatory processes in an organism. These molecules have essential functions in intercellular communication. Moreover, they have been used to diagnose and treat human diseases. To find novel bioactive molecules, traditionally, sequential column-chromatography has been used. However, there was inevitable limitation in the low abundance of the molecules of interest by low yield due to the many column steps.

To solve this problem, previously, the present inventors developed a new integrative method, Ligand Profiling and Identification (LPI), for searching various endogenous ligands. This method, based on parallel column chromatography methods and sensitive MS analysis, is suitable for searching low abundance bioactive molecules rapidly and simultaneously. Recently, for the efficient purification, we evolved this LPI technology by adding the protease filtering method. We assumed that these systematic and sensitive analytical techniques could be effectively used for the identification of novel bioactive molecules from tissues or body fluids.

These prior art references do not specifically describe or suggest combining an insulin sensitizer with an anorectic, and effects of such combination. Development of excellent drugs which are sufficiently improved as a medicine having an excellent diabetic treatment effect without apparent detection of side effects is desired.

### SUMMARY OF THE INVENTION

In the present invention to find novel ligand which could stimulate glucose uptake in 3T3-L1 adipocytes from serum, Lysophosphatidylcholine (LPC) was identified as a novel ligand which could activate glucose uptake. The present invention shows for the first time that LPC stimulates glucose uptake in 3T3-L1 adipocytes and lowers blood glucose level in diabetes model mice. Furthermore, this metabolic regulation of LPC requires activation of PKC δ.

In another aspect of the present invention, the role of peripheral urocortin was investigated in glucose homeostasis. UCN enhanced insulin induced phosphorylation of IR and the subsequent intracellular signaling in human insulin receptor-overexpressed Rat-1 cells (hIRcB cells) and C2C12 myotubules. Furthermore, being consistent with our in vitro findings, intravenous injection of UCN also sensitized insulin-induced down-regulation of blood glucose level in STZ mice. These findings showed for the first time that urocortin sensitized the insulin function through the mechanism of IR sensitization. Thus, the present invention screened endogenous peptides and we found urocortin as insulin sensitizer.

An object of the present invention is to provide a glucose uptake stimulator which comprises a compound selected from the group consisting of lysophosphatidylcholine, lysophosphatidylserine, lysophosphatidic acid, and urocortin. The lysophosphatidylcholine, lysophosphatidylserine, and lysophosphatidic acid activates a glucose uptake without insulin. Urocortin acts as co-factor for insulin action in the regulation of glucose homeostasis.

The lysophosphatidylcholine has no effects on Akt phosphorylation. The acyl chain of lysophosphatidylcholine has carbon number 14 to 16. Myristoyl LPC, palmytoyl LPC stimulated glucose uptake, whereas, stearoyl LPC did not stimulate glucose uptake in 3T3-L1 adipocytes several lysophospholipids were treated to 3T3-L1 adipocytes. Palmytoyl lysophosphatidylethanolamine (LPE), palmytoyl lysophosphatidylglycerol (LPG) and palmytoyl lysophosphatidylinositol (LPI) did not stimulate glucose uptake in 3T3-L1 adipocytes, suggesting that the head group of LPC may contribute to the structural selectivity in stimulation of glucose uptake by LPC in 3T3-L1 adipocytes.

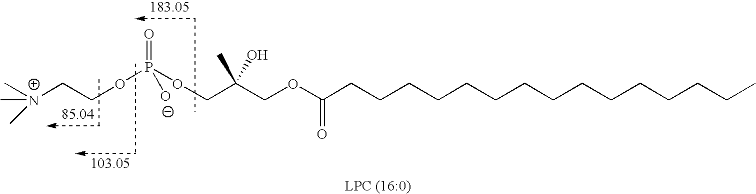

Another object of the present invention is to provide a pharmaceutical composition which comprises a compound selected from the group consisting of lysophosphatidylcholine, lysophosphatidylserine, lysophosphatidic acid, and urocortin. The pharmaceutical composition further comprises a pharmaceutically acceptable carrier, diluent or exipient. In addition, the pharmaceutical composition further comprises at least a compound selected from the group consisting of insulin secretion enhancers, biguanides, and α-glucosidase inhibitors.

A further object of the present invention is to provide a pharmaceutical composition where urocortin is used in combination with insulin.

A still object of the present invention is to provide a method for treating diabetes or diabetic complications in a mammal in need thereof, which comprises administering to said mammal an effecting amount of an insulin sensitizer selected from the group consisting of lysophosphatidylcholine, lysophosphatidylserine, lysophosphatidic acid, or urocortin. The diabetic complication is obesity, hyperlipidemia, arteriosclerosis, hypertension or heart disease. In addition, the method comprises a step of administering to said mammal an effecting amount of an insulin sensitizer in combination of at least a compound selected from the group consisting of insulin secretion enhancers, biguanides, and α-glucosidase inhibitors.

### BRIEF DESCRIPTION OF THE DRAWINGS

FIGS. 1A to 1E show an identification of a novel glucose uptake stimulating molecule from serum.

FIGS. 2A to 2D show the effects of LPC on the glucose uptake in 3T3-L1 adipocytes.

FIGS. 3A and 3B show LPC stimulating GLUT4 translocation in 3T3-L1 adipocytes.

FIGS. 4A and 4B show that LPC stimulates glucose uptake via PKCδ activation.

FIGS. 5A to 5E show anti-diabetic efficacy of intravenously administrated LPC in normal mouse and mouse Type I and II models of diabetes.

FIG. 6A show LPS specifically stimulating glucose uptake in 3T3-L1 adipocytes, and 6B show LPS stimulating glucose uptake in 3T3-L1 adipocytes dose-dependently.

FIGS. 7A and 7D show LPS lowering the level of blood glucose in normal mouse and Type I diabetes model mouse.

FIGS. 8A and 8B shows LPA stimulating glucose uptake in 3T3-L1 adipocytes with dose- and time-dependent manner.

FIGS. 9A and 9B shows LPA stimulating glucose uptake in 3T3-L1 adipocytes via LPA receptor and Gαi activation.

FIGS. 10A and 10B shows LPA stimulating glucose uptake in 3T3-L1 adipocytes by PI3-kinase dependent signaling pathway.

FIGS. 11A to 11D shows LPA lowering the level of blood glucose in normal mouse via LPA receptor activation

FIGS. 12A to 12D shown an effect of UCN on IR autophosphorylation in hIRcB cells.

FIGS. 13A and 13B show an effect of UCN on glucose uptake and IR phosphorylation in C2C12 myotubules.

FIGS. 14A and 14B show effects of UCN on plasma glucose control in normal and STZ-mouse.

### DETAILED DESCRIPTION OF THE EMBODIMENTS

An exemplary embodiment of the present invention will hereinafter be described in detail with reference to the accompanying drawings.

By a glucose uptake modulator is meant any agent which will lower blood glucose levels by increasing the responsiveness of the tissues to insulin.

By patients susceptible to insulin resistant hypertension is meant a patient who exhibits insulin resistance and is therefore likely to exhibit hypertension. Such patients are well known and readily determinable by a physician of ordinary skill in the art. By treatment is meant any lowering of blood pressure caused by insulin resistance and/or high circulating insulin levels. By prevention is meant partial to total avoidance of hypertension in insulin resistant patients, depending on the severity of the disease.

By “unit dose” is meant a discrete quantity of a glucose uptake modulator in a form suitable for administering for medical or veterinary purposes. Thus, an ideal unit dose would be one wherein one unit, or an integral amount thereof, contains the precise amount of glucose uptake modulator for a particular purpose, e.g., for treating or preventing obesity resulting from treatment with anti-diabetic drugs. As would be apparent to a person of ordinary skill in pharmaceutical formulations, glucose uptake modulator can be formulated into conventional unit doses. These unit doses can be packaged in a variety of forms, e.g., tablets, hard gelatin capsules, foil packets, glass ampules, and the like. Similarly, a unit dose may be delivered from a medicine dropper or from a pump spray. These various unit doses may then be administered in various pharmaceutically acceptable forms of liquid administration, i.e., orally or parenterally. Thus, for example, the contents of a foil packet may be dissolved in water and ingested orally, or the contents of a glass vial may be injected. Similarly, a discrete amount form such as a medicine dropper or a pump spray may be dissolved in water.

By “mammal” is meant any of a class (Mammalia) of higher vertebrates comprising man and all other animals that nourish their young with milk secreted by mammary glands and have the skin usually more or less covered with hair. Especially included in this definition are human beings, whose endurance, stamina or exercise capacity is less than optimal. Such human and non-human animals are readily diagnosed by a physician or veterinarian of ordinary skill.

Glucose homeostasis is maintained by the fine orchestration of hepatic glucose production and cellular glucose uptake. If our body fails to maintain glucose homeostasis, we can be under hyperglycemia or various metabolically disturbed conditions. In the search for novel factor which enhances glucose uptake in 3T3-L1 adipocytes, we applied new integrative method which is based on systematic parallel column chromatography, protease filtering and sensitive MS analysis and identified LPC.

We found that LPC stimulated glucose uptake with dose- and time-dependent manner. The stimulation of glucose uptake by LPC treatment is sensitive both to variation in the acyl chain lengths and difference in polar head group of LPC. Treatment of LPC to 3T3-L1 adipocytes resulted in significant increase the level of GLUT4 at the plasma membrane. The effects of LPC on glucose uptake are abrogated by the inhibitor of PKCδ, rottlerin, and expression of dominant negative PKCδ. Administration of LPC to mice resulted in significant lowering of blood glucose levels. Moreover, LPC improved the level of blood glucose in the mouse models of Type I diabetes (insulin-dependent diabetes) and type II diabetes (insulin-independent diabetes). These results suggest that LPC may lead to new insights into glucose homeostasis and a novel treatment modality for diabetes.

Lysophospholipids regulate variety of biological processes including cell proliferation, tumor cell invasiveness, and inflammation. LPC, produced by the action of Phospholipase A₂ (PLA₂) is a major plasma lipid component and transports fatty acids and choline to tissues. It is also known that LPC is highly related in the regulation of glucose homeostasis. Recently, it is has been shown that LPC enhances glucose-dependent insulin secretion from pancreatic-cells. One of LPC's reported physiological action is the induction of insulin secretion from pancreatic cells. Recently, Takatoshi et al. identified an orphan G-protein coupled receptor, GPR 119 as a novel Gs-protein coupled receptor for LPC. The GPR 119 is predominantly expressed in pancreatic cells and that activation of GPR 119 by LPC leads to glucose-dependent insulin secretion.

LPA has emerged as a potent and pleiotropic bioactive phospholipid known to regulate a number of cellular events via specific G protein-coupled receptors. LPA can regulate platelet aggregation, actin cytoskeleton activation, fibroblast proliferation, and neurite retraction. Two major pathways have been postulated for the extracellular production of LPA. As a first pathway LPA is released by activated platelets Second pathway: LPA is produced from lysophospholipids by autotaxin (lyso-PLD). Recently, it was reported that LPA is produced in the extracellular medium of adipocytes as the result of the secretion of autotaxin. LPA could be involved in the developmental control of adipose tissue which has key roles in regulating overall energy balance.

As a one of bioactive lysophospholipid, lysophosphatidylserine (LPS) is thought to be related in immunological regulation. However, the effects of LPS on cellular activities and the identities of its target molecules have not been fully elucidated. LPS has also been found in ascites of ovarian cancer patients. It has been reported to induce transient increases in intracellular calcium concentration in ovarian and breast cancer cell lines. LPS also stimulated interleukin-2 production in Jurkat T cells, showing inhibitory effect on Jurkat cell proliferation. Furthermore, LPS treatment enhanced nerve growth factor-induced histamine release in rat mast cells and nerve growth factor-induced differentiation of PC12 cells. Since limited reports have demonstrated the role of LPS in the modulation of some biological responses, its role in various cellular activities and its action mechanism should be investigated.

The present invention investigated the novel role of urocortin (UCN) as co-factor for insulin action in the regulation of glucose homeostasis. It has been well known that UCN acts as blood glucose enhancer. However, we found that UCN can sensitize the insulin-induced activation of signaling molecules, such as insulin receptor (IR), insulin receptor substrate (IRS) and protein kinase B (AKT) in IR over-expressed (hIRcB) cell and C2C12 myotubule. Interestingly, the effect of urocortin in vivo was different with dose in the regulation of blood glucose level. In the low dose (0.1 pM) of urocortin, it down-regulated blood glucose level and consequently increased IR phosphorylation in mouse skeletal muscle. In conclusion, we show the physiological phenomenon of urocortin which enhances insulin sensitivity, suggesting that urocortin may be useful to applying for therapeutic target of diabetes.

Urocortin is a 40 amino acid peptide as a member of the corticotrophin release factor (CRF) family. Urocortin is known for a principal hypothalamic factor in hypothalamic-pituitary adrenal (HPA) axis regulation. In addition, there is an increasing evidence for an additional important UCN role in energy balance regulation. UCN inhibit appetite and activates thermogenesis via catecholaminergic system, and gastric emptying and stimulates colonic motor function in various animal models. Recently there are some reports about UCN expression in peripheral tissue, such as skeletal muscle. But the role of peripheral UCN is still unknown in the regulation of glucose.

Lysophospholipids regulate variety of biological processes including cell proliferation, tumor cell invasiveness. LPC, produced by the action of phospholipase A₂ (PLA₂) on phosphatidylcholine, promotes inflammatory effects, including increased expression of endothelial cell adhesion molecules and growth factors, monocyte chemotaxis, and macrophage activation. For the first time, the present invention provides evidence that LPC is a blood borne hormone involved in glucose homeostasis. To find this molecule, we used new, integrative method which contains parallel column chromatography, protease filtering and highly sensitive MS analysis (Baek, M. C., et al., Proteomics 6, pp 1741-1749, 2006). Treatment of LPC induced a rapid stimulation of glucose uptake in 3T3-L1 adipocytes via PI 3-kinase independent, PKCδ activation. Furthermore, administration of LPC to mouse models of diabetes resulted in significant lowering in blood glucose levels. Besides LPC, many lysophospholipids (LPL) are known to have diverse physiological and pathological functions. However, there is no report that they are involved in regulation of glucose homeostasis. As an endogenous lipid which related in glucose metabolism, dehydroepiandrosterone (DHEA) has been reported. Although, recent studies have demonstrated that DHEA increases glucose uptake rates in adipocytes, there is no report that its effectiveness on animal model. Therefore, we suggest that LPC might be the first endogenous lipid which regulates the level of blood glucose in the diabetic models of mice as well as in normal mice.

For the finding of a novel active ligand, we previously devised new methodology named LPI which is based on the concept of parallel HPLC and active fraction profiling by MS analysis. The parallel HPLC is effective on identification of active molecules by increasing yields as described in previous report. In this work, we added protease filtering method to the parallel HPLC for the more effective purification. Protease is commonly used for protein mapping or protein identification, but the protease filtering method utilizes protease as a purification tool like a column chromatography. Especially, protease filtering is appropriate for exclusion of the inactive peptides which have similar physicochemical properties with active molecule. Although, the inactive peptides are not easily removed by common sequential chromatographies, cleavage of inactive peptides by protease treatment gives rise to the structural changes in inactive peptides and segregation from active molecule by next column chromatography. By combining this protease filtering method and parallel HPLC, we have devised a new ligand identification method and identified LPC with less effort. Therefore, this integrative method may be useful for searching various bioactive molecules, like an orphan GPCR study, with small amount of starting materials.

The stimulation of glucose uptake in 3T3-L1 adipocytes and blood glucose lowering in mice by LPC treatment are sensitive to variations in the acyl chain lengths of LPC. While palmytoyl LPC and myristoyl LPC enhanced glucose uptake in 3T3-L1 adipocytes, stearoyl LPC was ineffective on stimulating glucose uptake in 3T3-L1 adipocytes. When several lysophospholipids, which are structurally different only in polar head group from palmytoyl LPC, were treated to 3T3-L1 adipocytes, there was no stimulation of glucose uptake. This structural specificity of LPC is also confirmed in mouse models. These results suggest that both acyl chain length and phosphatidylcholine head group are critical for stimulation of glucose uptake in 3T3-L1 adipocytes and lowering the level of blood glucose in mice.

Based on the rapid onset and structural specificity in LPC action, the present inventor speculates that the biological activity of LPC may be explained by LPC binding to a specific LPC receptor at the cell surface. Several lysophospholipids have been reported to be ligand for this GPCR family. LPC was reported as a direct ligand that binds and activates G2A and GPR4. However, recently, it was reported that LPC can activate but dose not bind directly G2A and GPR4 in other independent studies. Thus it remains an open question as to whether LPC stimulates glucose uptake via directly binding to G2A and GPR4 or indirectly via another unknown pathway.

The involvement of PKCζ activation in promotion of glucose uptake in adipocytes and muscle cells has long been recognized, but PKCδ activation also controls glucose transport. The involvement of PKCδ in glucose transport activation was originally elucidated in studies using pharmacological agents and insulin. Stimulation of the translocation of GLUT4 to the plasma membrane and glucose uptake by insulin was inhibited by rottlerin in rat skeletal muscle cells. Moreover, overexpression of PKCδ induced the translocation of GLUT4 to the plasma membrane and increased basal glucose uptake to levels attained by insulin. In this study, LPC-induced enhancement of glucose uptake was blocked by rottlerin and the expression of dominant negative PKCδ. However, the pretreatment of conventional PKC inhibitor Gö6976 or the expression of dominant negative PKCζ was shown to have no effect on LPC-stimulated glucose uptake. These findings suggest that only PKCδ is essential for the LPC-stimulated glucose uptake.

One of LPC's reported physiological action is the induction of insulin secretion from pancreatic β-cells. Recently, Takatoshi et al. identified an orphan G-protein coupled receptor, GPR 119 as a novel Gs-protein coupled receptor for LPC (Soga, T., et al., Biochem Biophys Res Commun 326, pp 744-751, 2005). The GPR 119 is predominantly expressed in pancreatic β-cells and that activation of GPR 119 by LPC leads to glucose-dependent insulin secretion. In this study, we administrated LPC to mice under fasting condition. We also observed that there is no change in concentration of serum insulin after LPC administration to mice. These suggest that the blood glucose lowering in mice is not mediated by insulin secretion but by the direct function of LPC after LPC stimulation.

In summary, our present study shows that LPC stimulate glucose uptake in 3T3-L1 adipocytes. This effect is mediated by PI 3-kinase independent, PKCδ dependent signaling pathway. Moreover, LPC directly lowers the level of blood glucose in diabetic mice models. This new discovery of the blood glucose lowering function of LPC may shed new light on glucose homeostasis and other aspects of glucose metabolism-related biology. The relationship between LPC and metabolic syndrome also merit further investigation. Finally, our results raise the high possibility that LPC may be a useful target for the development of drug therapies for diabetes.

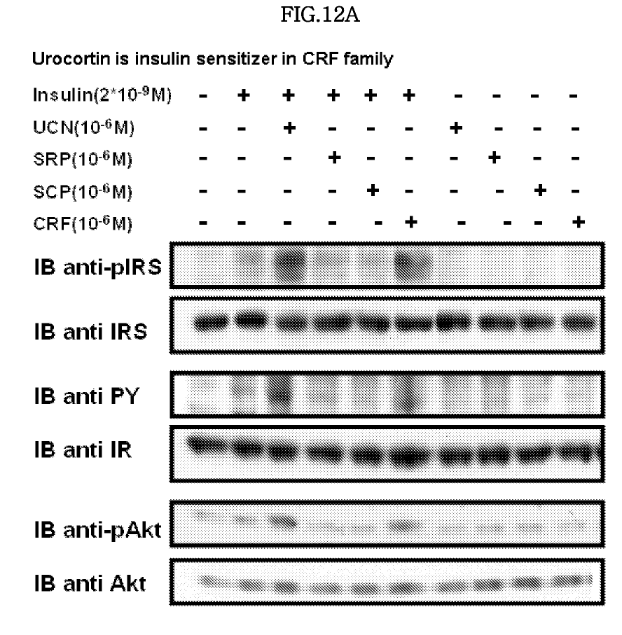

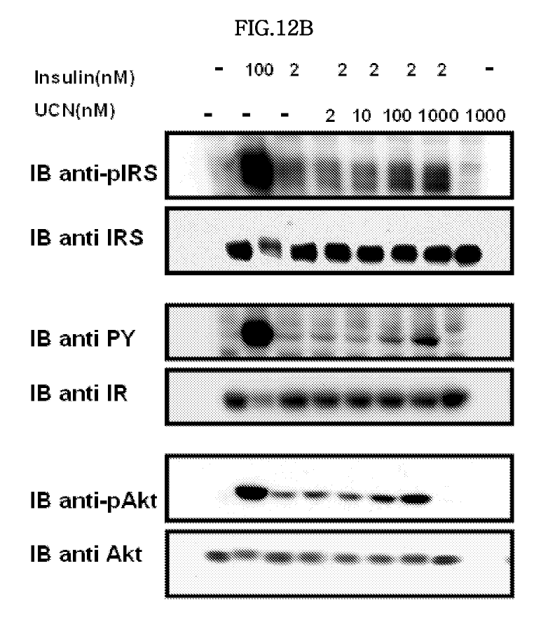

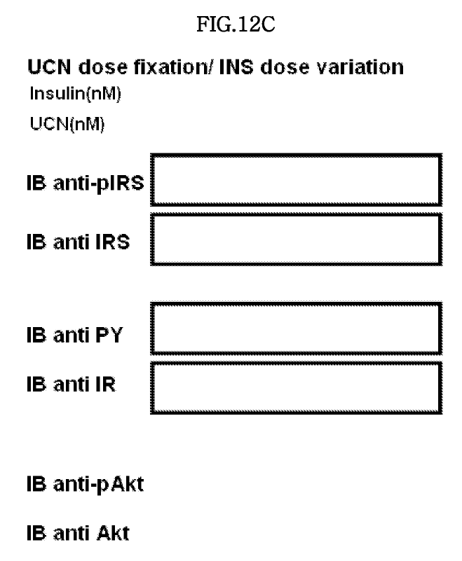

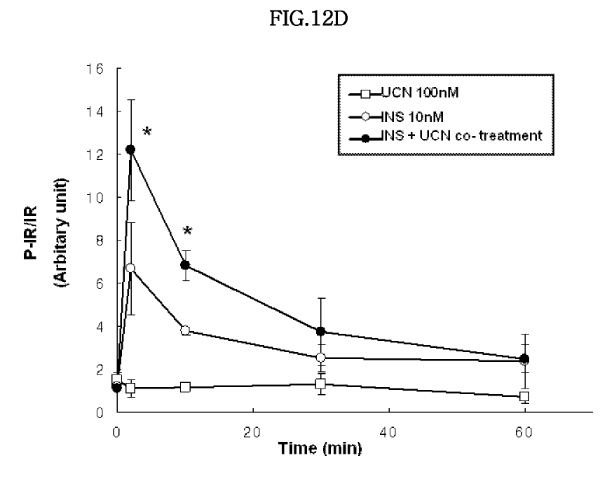

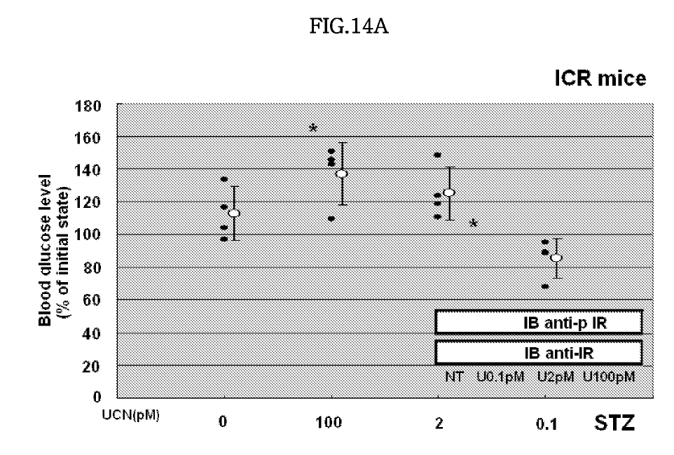

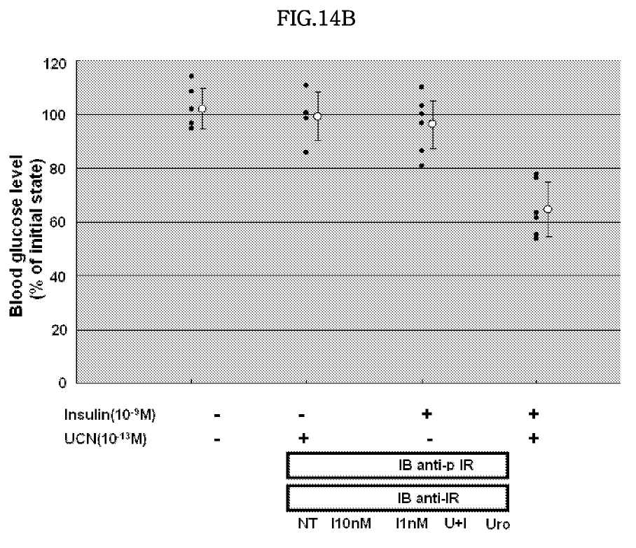

UCN has been known as blood glucose enhancer, but, in the present invention, blood glucose level was down-regulated by injection of UCN in normal ICR mouse (FIG. 14A) and further down-regulated by co-injection of insulin and UCN, compared to insulin alone, in streptozotocin (STZ)-mouse (FIG. 14B). Moreover, the present invention investigated the molecular mechanism of UCN-mediated down-regulation of blood glucose level. The present inventor found that UCN sensitized the insulin-mediated IR phosphorylation, implicated to IR activation, in IR-overexpressed (hIRcB) and differentiate C2C12 myotubules (FIG. 12, 13). And these effects were connected to glucose uptake in C2C12 myotubules. This is the first finding that GPCR ligand specifically sensitizes insulin-induced IR activation and physiological function, glucose regulation.

Insulin has been known as major glucose regulator in blood. However, for efficient and fine regulation of blood glucose level, it has been suggested to be need of co-factors for insulin functions. These co-factors may have different functional weights between physiological and pathological conditions. In normal physiological condition, insulin has a role as major glucose regulator and so the co-factors may be aside in the regulation of glucose homeostasis. But in pathological condition, such as diabetes and obesity, the insulin action is highly attenuated and the co-factors may have a great portion of glucose regulation by enhancing insulin action.

UCN showed the hypoglycemic effect, even though in the insignificant number of mouse and the cooperative effect in the glucose regulation with insulin in STZ-mouse. Therefore, there are possibilities that UCN may have more potent effect in glucose regulation in pathological condition. In conclusion, the present invention revealed the novel mechanism of insulin-mediated glucose regulation and the novel function of UCN. It is interesting that IR activity can be regulated by GPCR and UCN have shown opposite functions between CNS and peripheral system in the aspect of glucose homeostasis.

A pharmaceutical composition of the present invention can be used as an agent for preventing or treating diabetes or diabetic complications. Examples of the diabetes include insulin-dependent diabetes mellitus, insulin-independent diabetes mellitus and etc. Further, a pharmaceutical composition of the present invention can be used as an agent for preventing or treating diabetic complications (e.g., neuropathy, nephropathy, retinopathy, macroangiopahty, coronary artery diseases, osteopenia, etc.). Further, a pharmaceutical composition of the present invention can be used as an agent for treating impaired glucose tolerance.

Further, use of a pharmaceutical composition of the present invention in combination with insulin secretion enhancers, biguanides, α-glucosidase inhibitors, and etc. provides a more excellent blood sugar lowering effect.

Dosage forms of a pharmaceutical composition of the present invention or its respective active ingredients include oral dosage forms such as tablets, capsules (including soft capsules and microcapsules), powders, granules, syrups, and etc.; and non-oral dosage forms such as injections (e.g., subcutaneous injections, intravenous injections, intramuscular injections, intraperitoneal injections, etc.), external application forms (e.g., nasal spray preparations, transdermal preparations, ointments, etc.), suppositories (e.g., rectal suppositories, vaginal suppositories, etc.), pellets, drip infusions, and etc.

The dosage of a pharmaceutical composition of the present invention may be appropriately determined with reference to the dosage recommended for the respective drug(s), and can be selected appropriately according to the subject, the age and body weight of the subject, current clinical status, administration time, dosage form, method of administration, combination of the drug(s), and etc. The dosage of an insulin sensitizer and an anorectic can be selected appropriately based on clinically used dosage. For administration of an insulin sensitizer to an adult diabetic patient (body weight: 50 kg), for instance, the dose per day is usually 0.01 to 1000 mg, preferably 0.1 to 500 mg. This dose can be administered once to several times a day.

The present invention is further explained in more detail with reference to the following examples. These examples, however, should not be interpreted as limiting the scope of the present invention in any manner.

### EXAMPLE

Method & Material

Materials: Synthetic 14:0, 18:0, 18:1 LPC, insulin and streptozotocin (STZ) were obtained from Sigma (St. Louis, Mo.). Other lysophospholipids were purchased from Avanti polar lipids. All lipids were dissolved in MeOH as a 50 mM stock. All lipid stocks were stored under nitrogen at −70° C. in glass vials as single-use aliquots and used within a month. Gö6976 and rottlerin were from Calbiochem. Antibodies were purchased from the following sources: Polyclonal anti-GLUT4 antibody was from Biogenesis Ltd (Sandown, N.H.). Anti-phospho-Ser473 AKT1 antibody was from Sigma. Anti-phospho-Tyr989 IRS1 was produced in our laboratory. [¹⁴C] 2-deoxy-D-glucose (300 mCi/mmol) was purchased from Moravek Biochemicals. Trypsin was from Roche (Mannheim Germany). Tissue culture media and fetal bovine serum were obtained from GIBCO. All other reagents were of analytical grade.

Cell culture: 3T3-L1 fibroblasts were grown to confluence in DMEM containing a high glucose concentration, 10% fetal bovine serum, 50 U of penicillin per ml, and 50 ug of streptomycin per ml and maintained in a 5% CO₂ humidified atmosphere at 37° C. 3T3-L1 was induced to differentiate into adipocytes, as described previously (van den Berghe, N., et al., Mol Cell Biol 14, pp. 2372-2377, 1994).

Animals. Male ICR mice were purchased from hyochang science (ROK). C57BLKSJ-db/db mice were purchased from SLC (Japan). After intravenous injection of LPC, blood glucose was measured regularly with a portable glucose meter (Gluco-Dr, ROK) after tail snipping. For measurement of serum insulin, blood samples of mice were determined with the insulin-RIA Kit (LINCO, Mo.). Insulin deficient mice were induced in male ICR mice by two consecutive daily intraperitoneal injection of STZ (200 mg/kg) dissolved in sodium citrate (pH 5.5). On the third day after the last STZ injection, acute glucose lowering effect was analyzed after intravenous injection of vehicle, LPC or insulin as described above.

HPLC purification. Approximately 350 ml of fresh human serum was mixed with 70% (v/v) acetone, 1 M acetic acid, and 20 mM HCl and was centrifuged at 20,000 g for 30 min at 4° C. The resultant supernatant was collected and extracted three times with diethyl ether. The aqueous phase was centrifuged at 20,000 g for 30 min at 4° C., and the supernatant was loaded onto cartridges of SepPak C18 (Waters) for pre-clearing. Eluent was directly loaded onto a C18 reverse-phase HPLC column (Vydac 218TP1022, 22 mm×250 mm). 10 ml fractions were collected, and ˜1% of each fraction was assayed for glucose uptake in 3T3-L1 adipocytes. The active fractions were trypsinized for 12 hr at 37° C. and applied with equal amount to a C4 reverse-phase HPLC column (Vydac 214TP5215, 2.1 mm×150 mm) and a cation-exchange HPLC column (Amersham Pharmacia Min-S HR 5/5, 4.6 mm×50 mm) each.

Mass spectrometry and data analysis. ESI-MS and tandem mass spectrometry (MS/MS) analyses were performed using QSTAR PULSAR I hybrid Q-TOF MS/MS (Applied Biosystems/PE SCIEX, Toronto, Ontario) equipped with a nano-ESI source. The samples were dissolved in 0.1% trifluoroacetic acid delivered into the ESI source using a protana nanospray tip (Odense, Denmark). All of the masses detected by QSTAR were calculated using Analyst QS software provided by Applied Biosystems (AB). The QSTAR was operated at a resolution of 8,000-10,000 with a mass accuracy of 10-30 ppm using external calibration maintained for 24 h. The voltage of the spray tip was set at 2300V. To identify the common mass by mass information, combined online-database; Dictionary of Natural Products (Chapman &Hall/CRC) was used.

Glucose uptake measurement. For measuring glucose uptake in 3T3-L1 adipocytes, cells were grown in serum-free DMEM for 16 h and then incubated in the absence or presence of insulin or lysophospholipids for the indicated times at 37° C. Uptake was measured by adding 1 μCi of [¹⁴C] 2-deoxy-D-glucose and 3 mM 2-deoxy-D-glucose. After 10 min, the assay was terminated by two quick washes with ice-cold PBS. Cells were lysed in 0.5 ml of lysis buffer containing 0.5 N NaOH and 0.1% SDS. The cell lysates were used for liquid scintillation counting and nonspecific uptake was assayed in the presence of 10 μM cytochalasin B(van den Berghe, N., et al., Mol Cell Biol 14, pp. 2372-2377, 1994).

Membrane fractionation of adipocytes. For obtaining total membranes (TM) from 3T3-L1 adipocytes, cells were collected into 10 ml of ice-cold HES buffer (250 mM sucrose, 1 mM EDTA, 1 mM phenylmethylsulfonyl fluoride [PMSF], 1 μM pepstatin, 1 μM aprotinin, 1 μM leupeptin, and 20 mM HEPES, pH 7.4) and subsequently homogenized with 30 strokes in a glass Dounce homogenizer at 4° C. After centrifugation at 1,000 g for 5 min at 4° C. to remove unbroken cells, the supernatant was then centrifugated at 212,000 g for 90 min at 4° C. to yield a pellet of total cellular membranes. To obtain the plasma membrane (PM) subcellular fraction from 3T3-L1 adipocytes, differential ultracentrifugation was used as described previously (Perrini, S., et al., Diabetes 53, pp. 41-52, 2004).

Adenoviral transfection of PKC isoforms. The adenovirus expression vector for PKCδ or ζ recombinant adenoviruses has been described previously. After differentiation of cultured 3T3-L1 adipocytes, the culture medium was aspirated and culture was infected with the viral medium containing PKCδ or ζ recombinant adenoviruses for 24 h. The cultures were then washed twice with DMEM and refed. Cells 48 h post-infection were used for glucose uptake or Immunoblotting.

Immunoblotting. For preparing total cell lysates, 3T3-L1 adipocytes were washed with Ca²⁺/Mg²⁺-free PBS and then lysed in the lysis buffer (50 mM HEPES, pH 7.2, 150 mM NaCl, 50 mM NaF, 1 mM Na₃VO₄, 10% glycerol, 1% Triton X-100). The lysates were centrifuged at 15,000 rpm for 15 min at 4° C. The proteins were denatured by boiling in laemmli sample buffer for 5 min at 95° C., separated by SDS-PAGE. SDS-gel was transferred to nitrocellulose membrane using Hoefer wet transfer system. Membranes were blocked in TTBS (20 mM Tris-HCl, pH 7.6, 150 mM NaCl, 0.05% Tween 20) containing 5% skimmed milk powder for 30 min and then incubated with antibodies for 3 hours. After washing membranes several times with TTBS, the blots were incubated with HRP (Horseradish peroxidase)-conjugated goat anti-rabbit for 1 hour. The blots were washed with TTBS and developed by ECL.

Statistical analysis. All data are expressed as mean±SE. Statistical analysis was performed by Student's t test. *P<0.01 was considered to indicate statistical significance.

### EXAMPLE 1

### Identification of Lysophosphatidylcholine as a Glucose Uptake Stimulating Molecule from Human Serum in 3T3-L1 Adipocytes

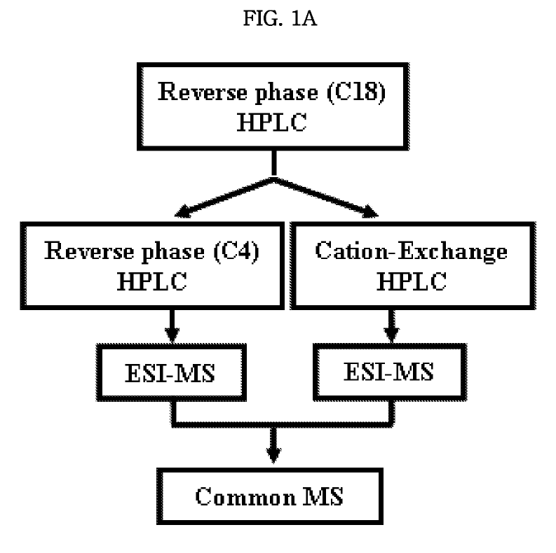

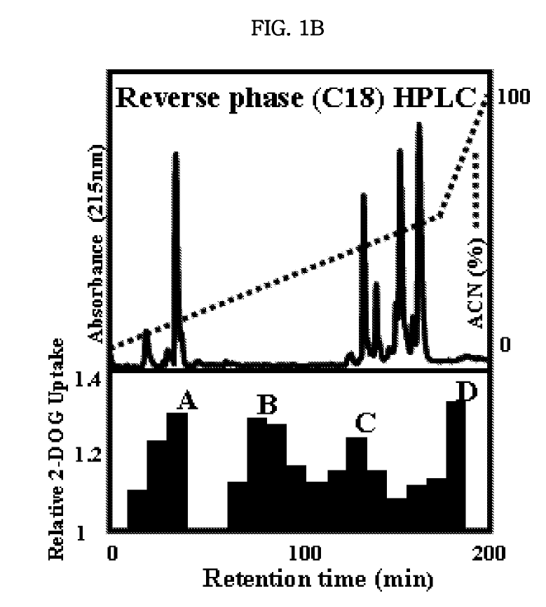

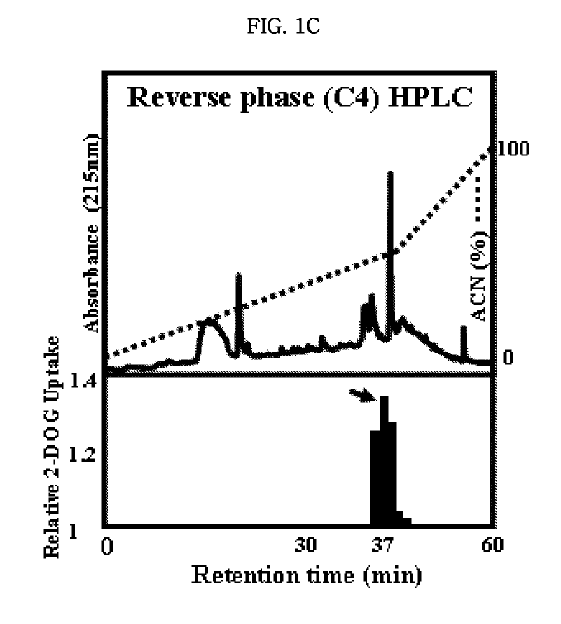

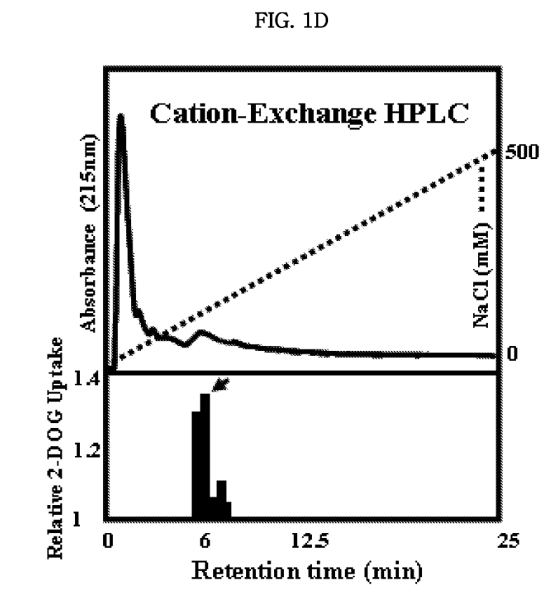

To investigate endogenous factors which stimulate glucose uptake in 3T3-L1 adipocytes, we used a new integrative method which is based on systematic parallel column chromatography, protease filtering method and sensitive MS analysis (FIG. 1A). The fundamental principle of parallel HPLC is that it uses profiling analysis to identify target molecules instead of traditional, sequential purification (Baek, M. C., et al., Proteomics 6, pp, 1741-1749, 2006). Low yield by multi-step, sequential columns is a critical limitation of purification because yields after each column are reduced exponentially as purification progresses. This new method minimizes the sequential HPLC steps and utilizes partially purified HPLC fraction for the identification of target molecules, only small amounts of starting material is required compared to the multi-step, sequential HPLC.

In addition to parallel HPLC, we used protease filtering method for efficient purification. If an active fraction doesn't lose its activity after treatment of specific protease, then we can get the fraction which contains active molecule and is drastically separated from various inactive peptides by following column chromatography. Therefore, this method is useful for the purification of non-peptide molecules such as lipids, amines and carbohydrates. With this new integrative method, first, we fractionated acetone extract from human serum (350 ml) by C18 reverse phase (C18) HPLC. Then these HPLC fractions were treated to 3T3-L1 adipocytes and glucose uptake was measured by determining the increase of [14C] 2-deoxy-D-glucose uptake (van den Berghe, N., et al., Mol Cell Biol 14, pp. 2372-2377, 1994).

As shown in FIG. 1B, there were at least four kinds of active fractions (A-D) and we tested if their activities are reduced by trypsin treatment. Only the activity of fraction D was not influenced by trypsin treatment, so the fraction D is trypsinized and further separated by C4 reverse phase (C4) and Cation-exchange (SCX) HPLC in parallel. All the fractions of C4 and SCX are screened by measuring the glucose uptake from 3T3-L1 adipocytes (FIGS. 1C and 1D).

The active fractions from each column (37 min from C4, 6 min from SCX) are analyzed by ESI-QTOF mass spectrometer. To find common mass, each mass spectrum was compared and there was only one common mass value of 495.33 as a monoisotopic mass (FIG. 1E-upper panel and FIG. 1E-middle panel). With this mass information, we searched combined online-database (Dictionary of Natural Products) and identified as palmytoyl lysophosphatidylcholine (LPC). To confirm whether the target molecule is LPC, we analyzed the each fragmentation pattern of standard LPC and 495.33 mass in MS/MS spectrum (FIG. 1F). The standard LPC product-ion spectrum in the positive-ion mode displays several ions originated from the collision-induced dissociation of the phosphocholine head group, including the most intense peak at m/z 183 (FIGS. 1F-bottom panel and 1G). The fragmentation pattern of 495.33 mass from C4 and SCX was exactly matched with standard LPC (FIGS. 1F-upper panel and 1F-middle panel). Based on the physical properties stated above, we concluded that the active substance is a LPC.

FIGS. 1A to 1E show an identification of a novel glucose uptake stimulating molecule from serum. FIG. 1A shows schematic representation of identification strategy for the serum factor which can stimulate glucose uptake in 3T3-L1 adipocytes. FIG. 1B shows C18 reverse-phase HPLC (Vydac 218TP1022, 22 mm×250 mm) elution profile of the serum. Relative 2-deoxy-D-glucose uptake is expressed as a ratio of the increment obtained by each fraction treatment versus vehicle treatment in 3T3-L1 adipocytes. Active fraction D was arbitrarily selected and trypsinized for further purification. FIG. 1C shows C4 reverse-phase HPLC (Vydac 214TP5215, 2.1 mm×150 mm) elution profile of the fraction D. FIG. 1D shows a cation-exchange HPLC (Amersham Pharmacia Mini-S HR 5/5, 4.6 mm×50 mm) elution profile of the fraction D. FIG. 1E shows Mass analysis by ESI-TOF mass spectrometer. Mass spectrum of active fraction of FIG. 1C(top), FIG. 1D(middle) and standard palmytoyl (16:0) LPC (bottom). FIG. 1F shows a pattern analysis in mass fragmentation and MS/MS spectrum of 495.33 mass in each mass spectrum of FIG. 1E.

### EXAMPLE 2

### Effects of LPC on the Glucose Uptake in 3T3-L1 Adipocytes

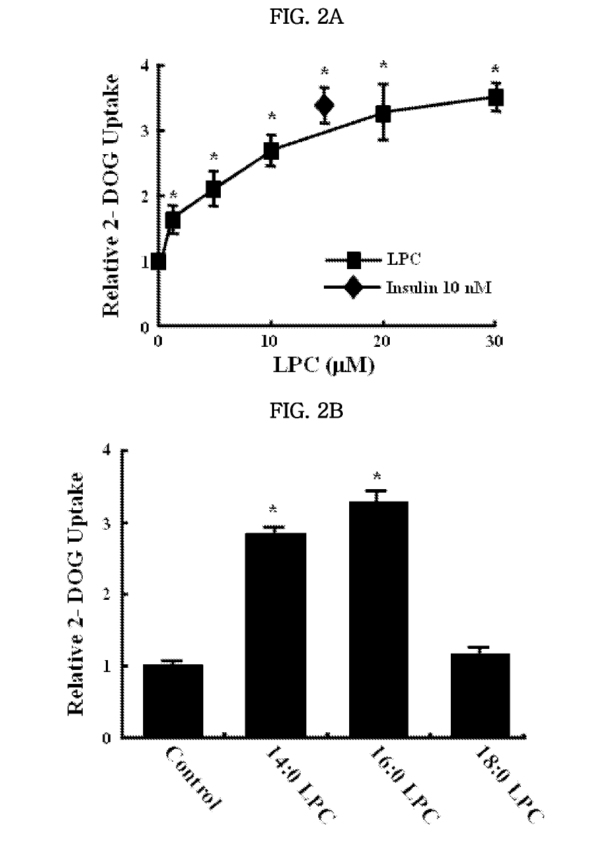

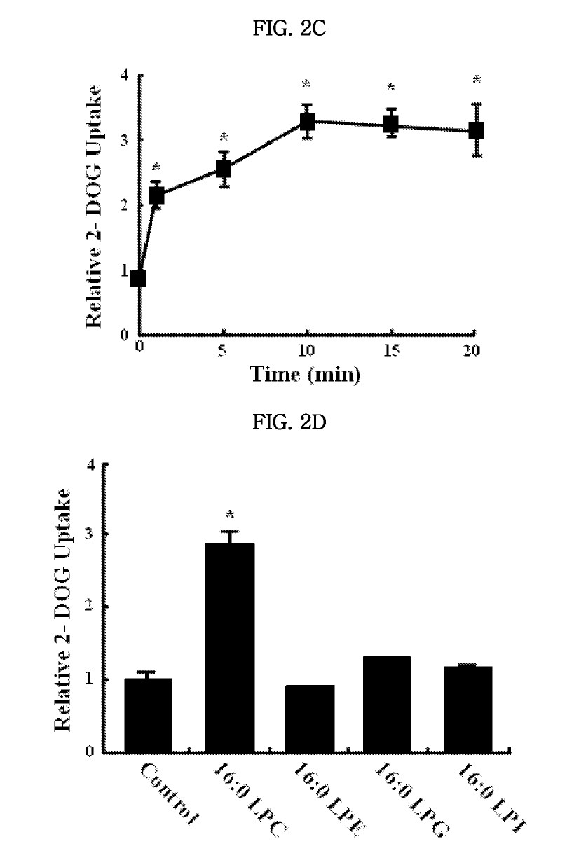

For investigating the effects of LPC on the glucose uptake, 3T3-L1 adipocytes were incubated in the presence of various concentrations of standard LPC for different times. LPC stimulated a time- and dose-dependent increase in glucose uptake in 3T3-L1 adipocytes. An initial statistically significant effect of LPC on glucose uptake was observed at the concentration of 1 μM and the maximal effect at 20 μM (FIG. 2A). With 20 μM LPC, glucose uptake was maximally increased after 10 min of incubation with LPC (FIG. 2B). This concentration of LPC was not cytotoxic and was below the critical micellar concentration of 40 to 50 μM (Chaudhuri, P., et al., Circ Res 97, 674-681, 2005).

It is known that the skeletal muscle plays a central role in glucose metabolism, and impairment in glucose metabolism in the skeletal muscle often results in diabetes (Petersen, K. F., et al., Am J Cardiol 90, 11G-18G, 2002; Beck-Nielsen, H., et al., Diabetologia 37, pp 217-221, 1994). Although this report mainly focuses on the 3T3-L1 adipocytes, we also found that LPC increased the rate of glucose uptake in a dose dependent manner in C2C12 muscle cells (data not shown). These results imply that LPC may play a role in glucose regulation in both adipocytes and muscle cells.

To determine whether variations in the acyl chain lengths of LPC could affect glucose uptake, several LPC species were tested. Interestingly, myristoyl LPC, palmytoyl LPC stimulated glucose uptake, whereas, stearoyl LPC did not stimulate glucose uptake in 3T3-L1 adipocytes (FIG. 2C). For assessing whether other lysophospholipids could enhance glucose uptake in 3T3-L1 adipocytes, several lysophospholipids were treated to 3T3-L1 adipocytes. As shown FIG. 2D, palmytoyl LPE, palmytoyl LPG and palmytoyl LPI did not stimulate glucose uptake in 3T3-L1 adipocytes, suggesting that the head group of LPC may contribute to the structural selectivity in stimulation of glucose uptake by LPC in 3T3-L1 adipocytes.

FIG. 2A to 2D show the effects of LPC on the glucose uptake in 3T3-L1 adipocytes. FIG. 2A shows 3T3-L1 adipocytes grown in six-well plates were equilibrated in glucose-free Krebs-Ringer buffer for 1 hr and incubated with LPC (0 to 30 μM) or insulin (10 nM) for 10 min. After these treatments, [¹⁴C] 2-deoxy-D-glucose uptake was measured for 10 min as described in Material Methods. FIG. 2B shows 3T3-L1 adipocytes were incubated with LPC (20 μM) for 0 to 20 min. FIGS. 2C and 2D show relative [¹⁴C] 2-deoxy-D-glucose uptake in 3T3-L1 adipocytes incubated in the absence (control) or presence of equimolar concentrations (20 μM) of myristoyl lysophosphatidylcholine (14:0 LPC), palmytoyl lysophosphatidylcholine (16:0 LPC), stearoyl lysophosphatidylcholine (18:0 LPC), palmytoyl lysophsophatidylethanolamine (16:0 LPE), palmytoyl lyso-phosphatidylinositol (16:0 LPI), palmytoyl lysophosphatidylglycerol (16:0 LPG) for 10 min. Values are mean±SE of three independent experiments performed in triplicate. *P<0.05 vs. basal.

### EXAMPLE 3

### LPC Stimulates GLUT4 Translocation in 3T3-L1 Adipocytes

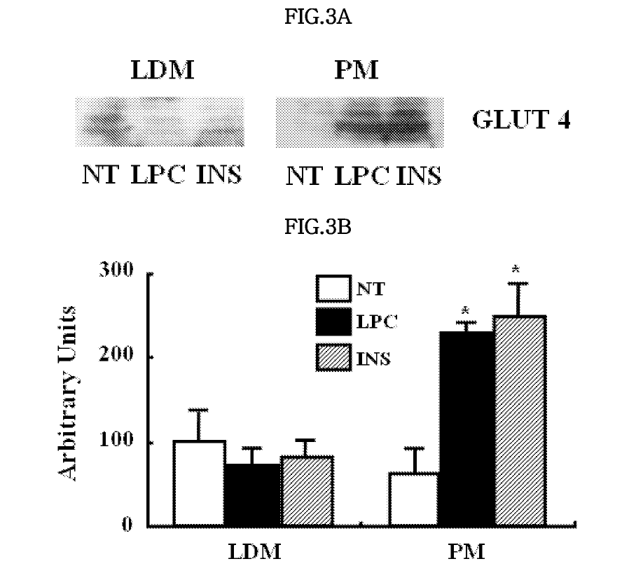

For assessing whether the ability of LPC to enhance glucose transport in 3T3-L1 adipocytes could be mediated by LPC-induced changes in the amounts of glucose transporter protein at the cell surface, the protein levels of GLUT4, the predominant glucose transporter isoforms expressed in 3T3-L1 adipocytes, was measured in PM fractions in the basal state or after treatment with LPC or insulin. LPC induced a significant increase in the PM content of GLUT4 proteins (180% of basal) like insulin (FIGS. 3A and 3B). The results suggest that both insulin and LPC stimulate GLUT4 translocation and are consistent with the observation in glucose uptake experiments.

FIGS. 3A and 3B show LPC stimulates GLUT4 translocation in 3T3-L1 adipocytes. A) Effect of insulin on GLUT4 translocation to the plasma membrane (PM) in 3T3-L1 adipocytes. Low-density microsome, LDM. 3T3-L1 adipocytes were stimulated for 10 min with 100 nM insulin or 20 μM lysophospholipids. In each experiment, the relative increase or decrease in the integrated density value (IDV) of GLUT4 after stimulation with compounds is calculated. B) Quantitation of relative increases is depicted. Values are mean±SE of three independent experiments performed in triplicate. *P<0.05.

### EXAMPLE 4

### LPC Stimulates Glucose Uptake Via PKCδ Activation

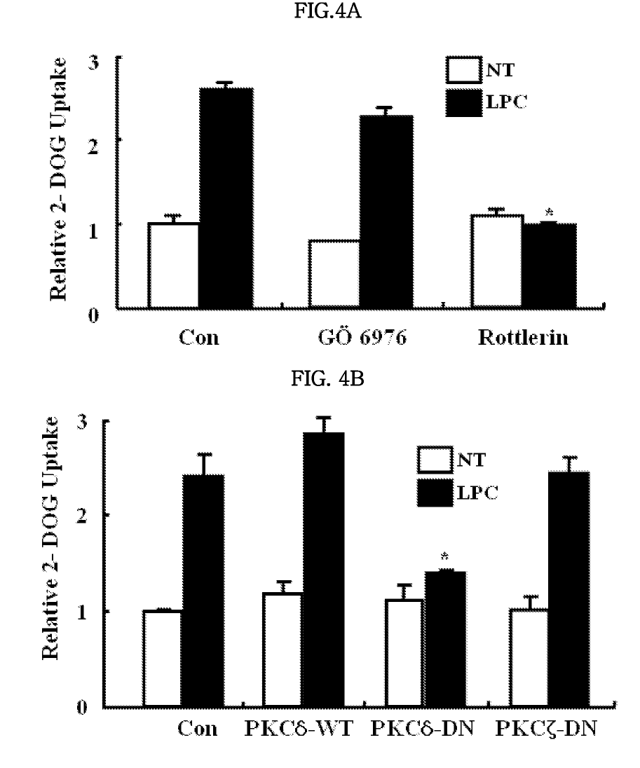

Insulin stimulation of glucose uptake in adiopocytes requires activation of IRS1, PI 3-kinase and subsequent activation of AKT (Burgering, B. M., et al., Nature 376, pp. 599-602, 1995; Baumann, C. A., et al., Nature 407, pp 202-207, 2000). Thus, to determine whether increased glucose uptake in response to LPC was associated with insulin dependent signaling pathway, IRS1 and AKT phosphorylation was checked. As expected, 10 nM insulin treatments of 3T3-L1 adipocytes resulted in augmentation of IRS1 and AKT phosphorylation (supplement data). By contrast, LPC treatment of adipocytes had no effects on phosphorylation of IRS1 and AKT (supplement data). Because LPC has been shown to activate conventional and novel PKC in various cells(Chaudhuri, P., et al., cell migration. Circ Res 97, 674-681, 2005), the involvement of these PKCs in the LPC-induced augmentation of glucose transport was assessed next. Pretreatment of 3T3-L1 adipocytes with 2 μM Gö6976, conventional PKC inhibitor, for 30 min did not alter LPC stimulation of glucose uptake. However, LPC-stimulated glucose uptake was completely inhibited by pretreatment with 10 μM rottlerin, an inhibitor of PKCδ (FIG. 4A)

To test the role of PKC more directly, we used an adenovirus expression system to overexpress specific PKC isoforms and dominant negative PKC isoforms in 3T3-L1 adipocytes. We assayed glucose uptake in 3T3-L1 adipocytes overexpressing the wild-type, dominant negative PKCδ or dominant negative PKCζ. The expression of the wild-type PKCδ induced slight increases in glucose transport activity of LPC-stimulated states, compared with that in control 3T3-L1 adipocytes. Expression of the dominant negative mutant of PKCδ reduced significant LPC-stimulated glucose transport activity. In contrast, overexpression of dominant negative PKCζ altered neither LPC-induced nor Insulin-induced glucose uptake (FIG. 4B). These findings demonstrate that a PKCδ could participate in LPC-induced glucose transport activation.

FIGS. 4A and 4B show that LPC stimulates glucose uptake via PKCδ activation. FIG. 4A shows 3T3-L1 adipocytes grown in six-well plates were equilibrated in glucose-free Krebs-Ringer buffer for 1 hr and were treated with 2 μM Gö6976, 10 μM rottlerin or buffer alone as indicated for 30 min. Then, cells were treated with vehicle (open bars) or 20 μM LPC (filled bars) for 10 min. After these treatments, [¹⁴C] 2-deoxy-D-glucose uptake was measured for 10 min as described in Material Methods. FIG. 4B shows expression levels PKCδ and PKCζ proteins. Lysates from control 3T3-L1 adipocytes and from those expressing PKCδ WT, PKCδ DN or PKCζ DN were immunoblotted with anti-PKCδ or PKCζ antibody (Top), and glucose uptake measurement in 3T3-L1 adipocytes (Bottom). Control 3T3-L1 adipocytes and 3T3-L1 adipocytes expressing PKCδ WT, PKCδ DN, or PKCζ DN were incubated with vehicle or 20 μM LPC or 10 nM insulin for 10 min. Values are mean±SE of three independent experiments performed in triplicate. *P<0.05.

### EXAMPLE 5

### Glucose-Lowering Effect of LPC in Mouse Models

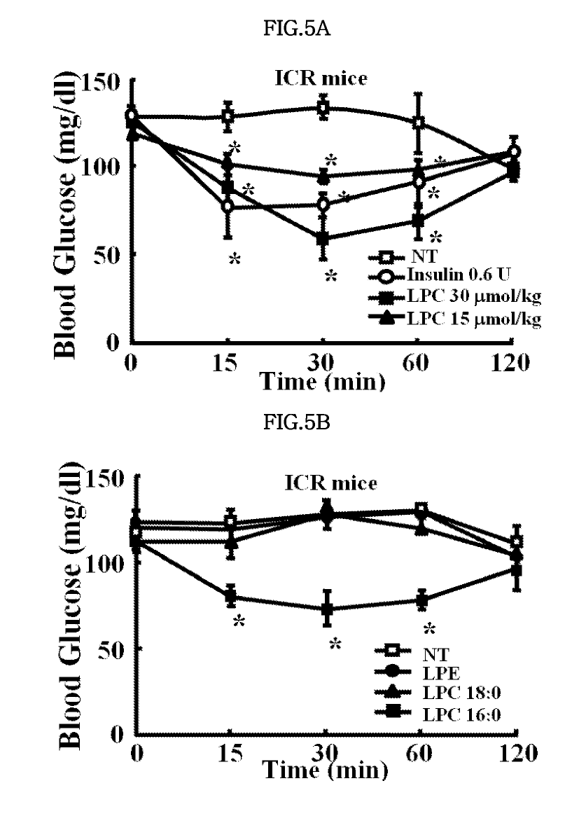

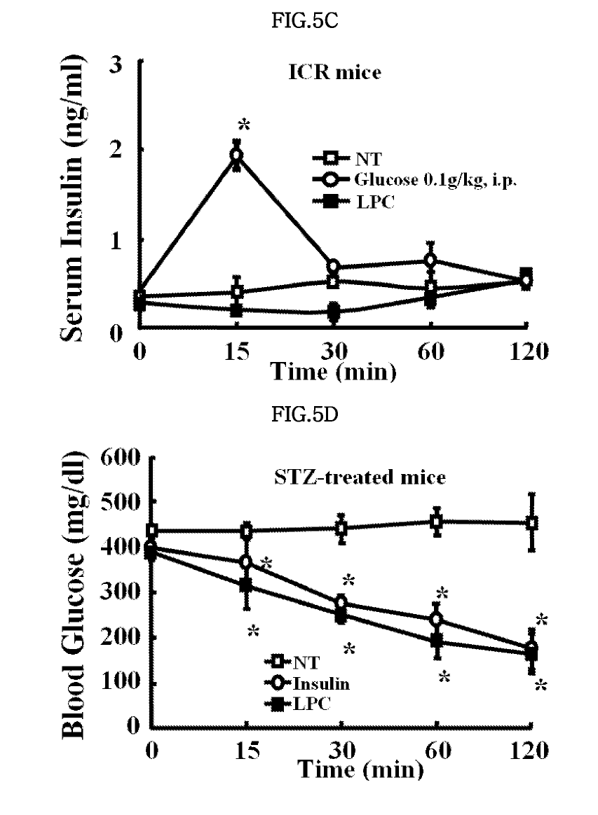

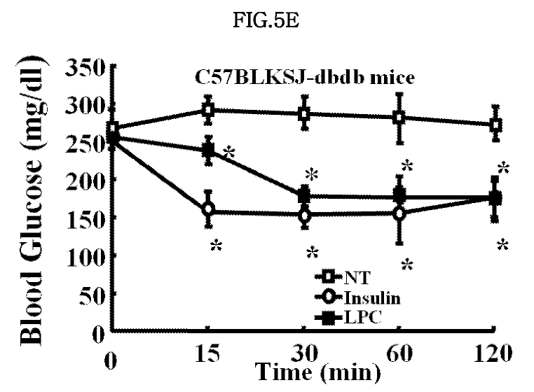

The in vivo effectiveness of LPC was examined in male, albino ICR (Institute of Cancer Research) mice. Acute administration of LPC (at 15 or 30 μmol/kg) to mice by intravenous (i.v.) injection resulted in a statistically significant fall in blood glucose levels within 30 min (FIG. 5A). This effect was dose-dependent and was not due to changes in blood insulin levels (FIG. 5C).

To determine whether different molecular species of LPC differ in their activities, LPC molecules with acyl chain of varying length or other lysophospholipid, lysophsophatidylethanolamine were administrated at doses equimolar to 30 μmol/kg (i.v.). Interestingly, only palmytoyl LPC had significant effect on the blood glucose lowering. (FIG. 5B). We next injected 30 μmol/kg (i.v.) LPC into streptozotocin (STZ)-treated insulin deficient mice. LPC significantly reduced blood glucose concentrations and the effect was similar to that induced by insulin injection (FIG. 5D).

Next, we investigated whether the injection of LPC also affected glycemia in insulin-resistant obese db/db mice. Upon injection of LPC, the blood glucose dropped to near normal levels (FIG. 5E). Taken together, these data suggest that LPC is able to regulate blood glucose level in both Type I and II diabetic mouse models as well as in normal mice.

FIG. 5A to 5E showed anti-diabetic efficacy of intravenously administrated LPC in mouse models of diabetes. FIGS. 5A and 5B showed that acute glucose lowering by LPC in ICR mice. Eight-week-old male mice were intravenously injected with PBS, insulin, LPC, or LPE. Blood glucose was monitored after dosing (0 to 120 min). FIG. 5C showed serum insulin level in eight-week-old male mice after single intravenous injection of LPC. FIG. 5D showed acute glucose lowering by LPC in streptozotocin (STZ)-treated insulin deficient ICR male mice. FIG. 5E showed acute glucose lowering by LPC in insulin-resistant obese C57BLKSJ-db/db mice. All animals had free access to water. Animal care was in accordance with institutional guidelines. All data are shown as means±SE (n=5-6). *P<0.05.

### EXAMPLE 6

### Effect of LPS on Glucose Uptake

6-1: Effects of LPS on the glucose uptake in 3T3-L1 adipocytes.

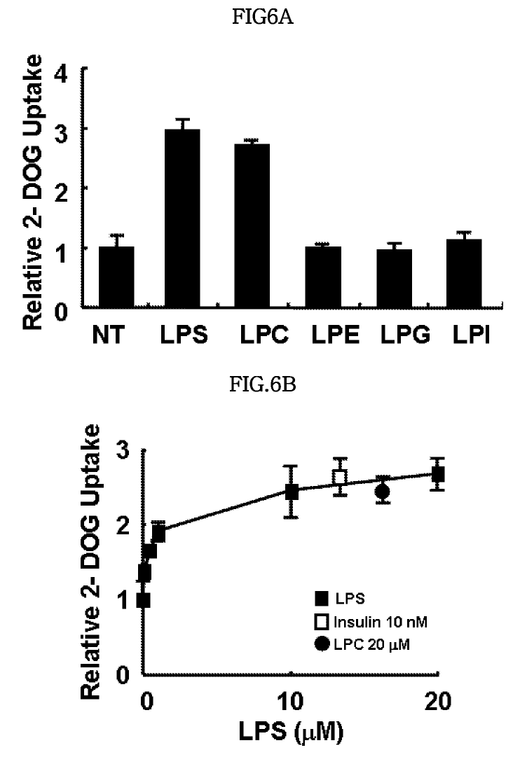

According to the substantially same method of EXAMPLE concerning LPC, the effect of LPS was tested in 3TS-L1 adipocytes, to show the result in FIGS. 6A and 6B.

3T3-L1 adipocytes grown in six-well plates were equilibrated in glucose-free Krebs-Ringer buffer for 1 hr and incubated with presence of equimolar concentrations (20 μM) of lysophosphatidylcholine (LPC), lysophosphatidylserine (LPS), lysophsophatidylethanolamine (LPE), lyso-phosphatidylinositol (LPI), lysophosphatidylglycerol (LPG) for 10 min. FIG. 6A shows that LPC and LPS specifically stimulated glucose uptake in 3T3-L1 adipocytes. FIG. 6B showed that LPS stimulate glucose uptake with dose dependent manner (0 to 30 μM). Values are mean±SE of three independent experiments performed in triplicate. *P<0.05 vs. basal.

6-2: Glucose lowering effects of LPS in diabetic mouse models.

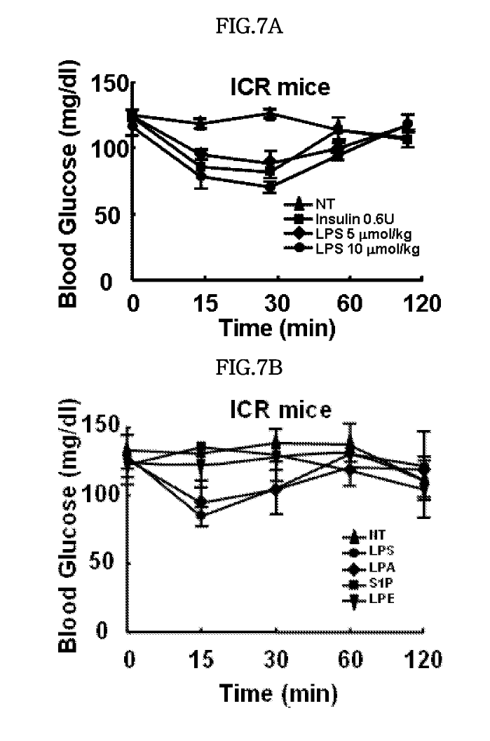

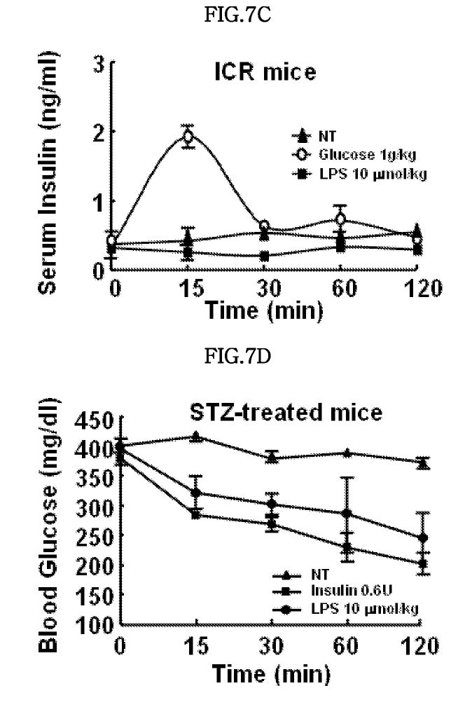

According to the substantially same method of EXAMPLE concerning LPC, the effect of LPS was tested in mouse, to show the result in FIG. 7A to 7D.

FIG. 7A to 7B showed glucose lowering efficacy of intravenously administrated LPS in diabetic mouse models. Eight-week-old male mice were intravenously injected with PBS, insulin, LPS. Blood glucose was monitored after dosing (0 to 120 min). As shown FIG. 7A) LPS lowered the level of blood glucose in normal mice dose- dependently. Other lysophospholipids such as SIP and LPE did not lower the blood glucose level in normal mice (FIG. 7B). Next, serum insulin level in eight-week-old male mice after single intravenous injection of LPS was measured. This effect was not due to changes in blood insulin levels (FIG. 7C). We next injected LPS into streptozotocin (STZ)-treated insulin deficient mice. LPS significantly reduced blood glucose concentrations and the effect was similar to that induced by insulin injection (FIG. 7D). From these data, LPS lowers the level of blood glucose dose-dependently and dose not affect insulin secretion. Furthermore, LPS has an effect on glucose regulation in insulin deficient, Type I diabetes model mouse. All animals had free access to water. Animal care was in accordance with institutional guidelines. All data are shown as means±SE (n=5-6). *P<0.05.

### EXAMPLE 7

### Effect of LPA on Glucose Uptake in 3T3-L1 Adipocytes

7-1: Effects of LPA on the glucose uptake in 3T3-L1 Adipocytes.

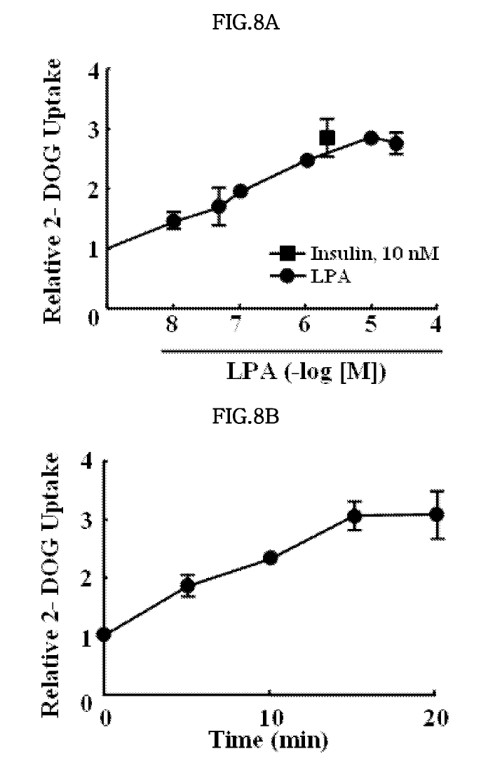

According to the substantially same method of EXAMPLE concerning LPC, the effect of LPA was tested in 3TS-L1 adipocytes, to show the result in FIGS. 8A and 8B.

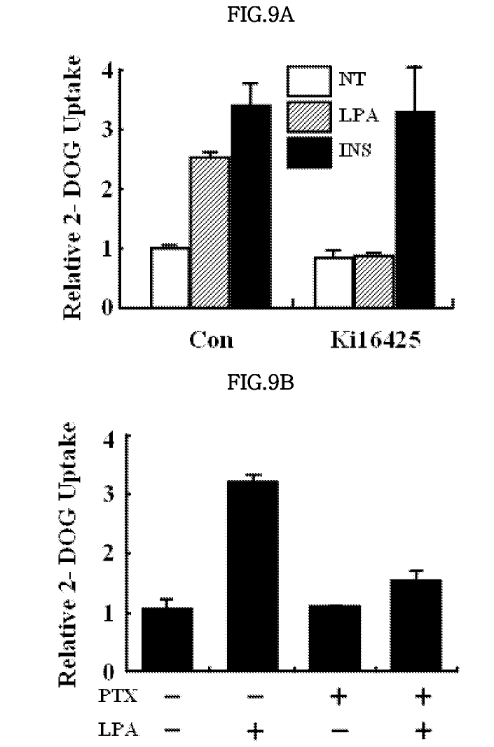

For investigating the effects of LPA on the glucose uptake, 3T3-L1 adipocytes were incubated in the presence of various concentrations of standard LPA for different times. LPA stimulated a time- and dose-dependent increase in glucose uptake in 3T3-L1 adipocytes. An initial statistically significant effect of LPA on glucose uptake was observed at the concentration of 1 μM and the maximal effect at 20 μM (FIG. 8A). With 20 μM LPA, glucose uptake was maximally increased after 10 min of incubation with LPA (FIG. 8B). FIG. 9A shows 3T3-L1 adipocytes grown in six-well plates were equilibrated in glucose-free Krebs-Ringer buffer for 1 hr and incubated with LPA (0 to 20 μM) or insulin (10 nM) for 10 min. After these treatments, [¹⁴C] 2-deoxy-D-glucose uptake was measured for 10 min as described in Material Methods. FIG. 8B shows 3T3-L1 adipocytes were incubated with LPA (20 μM) for 0 to 20 min.

7-2: Signaling mechanisms in the stimulation of glucose uptake by LPA

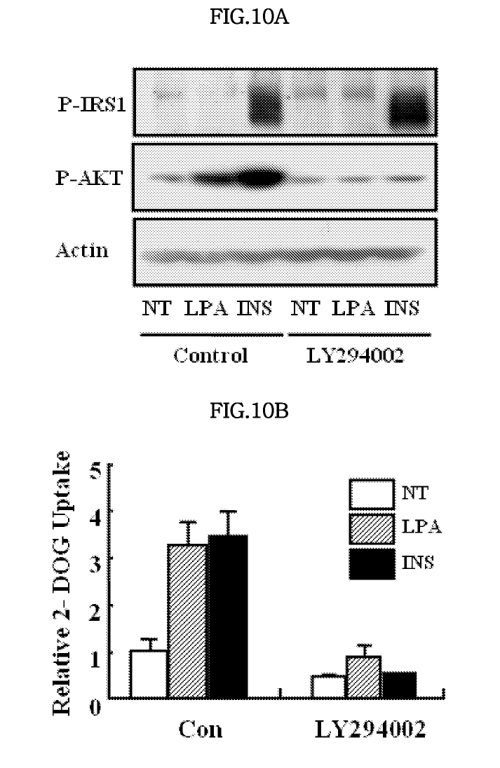

According to the substantially same method of EXAMPLE concerning LPC, the effect of LPA was tested in 3T3-L1 adipocytes, to show the result in FIGS. 9A, and 9B, and FIGS. 10A and 10B.

To investigated whether LPA affect glucose uptake via its receptor association we used LPA receptor antagonist, Kil6425. FIG. 9A shows that glucose uptake stimulation by LPA is completely inhibited by Kil6425 pretreatment. Next, we check whether LPA activates LPA receptor which coupled to Gαi by using the Gαi inhibitor, pertussis toxin.

FIG. 9B shows that LPA stimulates glucose uptake via to Gαi activation.

It is well reported that insulin stimulated glucose uptake via PI3-kinase dependent signaling pathways. To investigate whether LPA enhances glucose uptake via PI3-kinase dependent signaling pathway, we checked Akt, the down stream signal of PI3-kinase, is affected by LPA treatment. FIG. 10A shows that LPA stimulated Akt phosphorylation. This phosphorylation is inhibited by PI3-kinase inhibitor, LY294002 pretreatment. Next, to test LPA actually stimulates glucose uptake via PI3-kinase signal pathway, we pretreated LY294002, and measured glucose uptake in 3T3-L1 adipocytes. FIG. 10B shows that the stimulation of glucose uptake by LPA is dependent on PI3-kinase activation.

7-3: Glucose-lowering effect of LPA in mouse models.

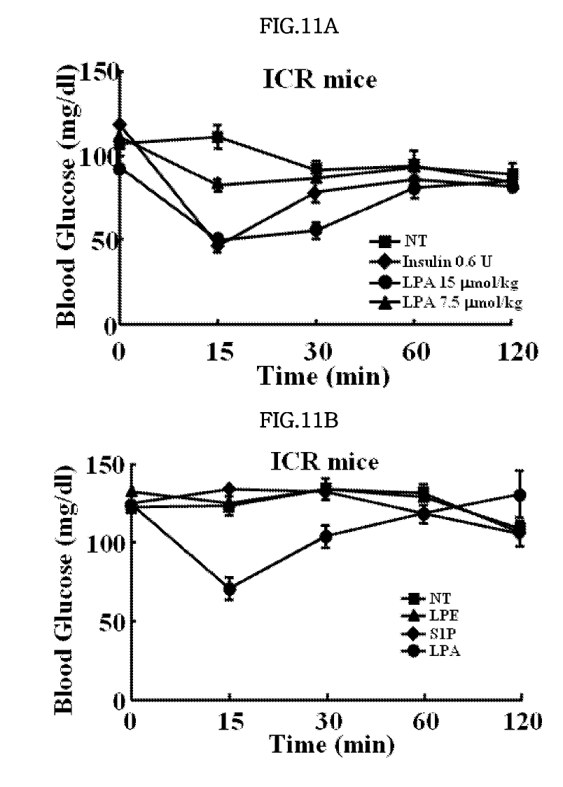

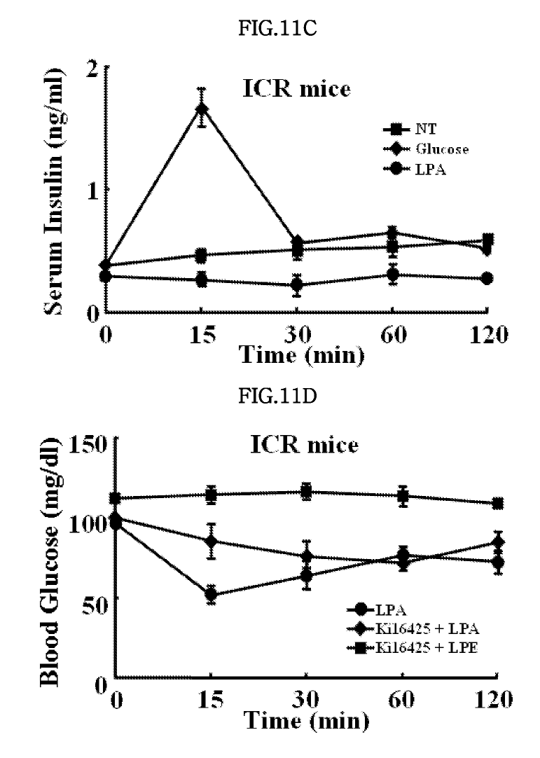

According to the substantially same method of EXAMPLE concerning LPC, the glucose-lowering effect of LPA was tested in mouse models, to show the result in FIG. 11A to 11D.

FIG. 11A to 11B showed glucose lowering efficacy of intravenously administrated LPA in mouse models. Eight-week-old male mice were intravenously injected with PBS, insulin, LPA. Blood glucose was monitored after dosing (0 to 120 min). FIG. 11A showed serum insulin level in eight-week-old male mice after single intravenous injection of LPA. LPA lowered the level of blood glucose in normal mice dose-dependently. Other lysophospholipids such as SIP and LPE did not lower the blood glucose level in normal mice (FIG. 11B). This effect was not due to changes in blood insulin levels (FIG. 11C).

Finally, we tested whether the glucose lowering effect by LPA is dependent on LPA receptor activation. Prior to administration of LPA, the LPA receptor inhibitor, Kil6425 was injected. FIG. 11D shows the glucose lowering effect by LPA is inhibited by LPA receptor inhibitor. From these data, LPA lowers the level of blood glucose dose-dependently and dose not affect insulin secretion. Furthermore, blood glucose lowering by LPA is mediated by LPA receptor activation. All animals had free access to water. Animal care was in accordance with institutional guidelines. All data are shown as means±SE (n=5-6). *P<0.05.

### EXAMPLE 8

### Effect of UCN on IR Autophosphorylation in hIRcB Cells

Materials: Dulbecco's modified Eagle's medium (DMEM) was purchased from BioWhittaker. Fetal bovine serum (FBS) and equine serum (ES) were from HyClone (Logan, Utah). Corticotrophin releasing factor (CRF), urocortin (UCN), stresscorpin relating peptide (SRP) and stresscorpin were synthesized from Anygen (Kwangju, Korea). Phospho-insulin receptor antibody, IRS antibody, IR antibody and AKT antibody were from cell signaling technology Inc. (Beverly, Mass.). [14C] 2-deoxy-glucose was purchased from moravek (Mercury, Calif.). All other chemicals were obtained from Sigma (St. Louis, Mo.).

All experiments, including the immunoblots, were independently repeated a minimum of three times. All immunoblots presented are representative of more than three separate experiments. Quantitative data are expressed as the means±S.E. Student's t tests were used where appropriate. A probability of p<0.05 was considered statistically significant.

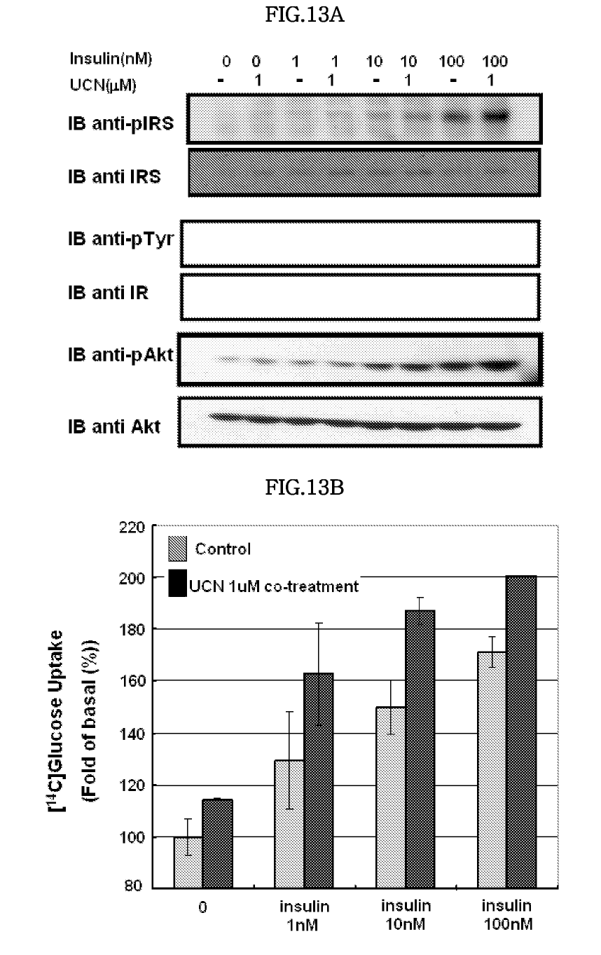

FIG. 12A shown a comparison of insulin sensitizing effect among CRF family which was obtained by incubating cells with 1 μM CRF family and/or 2 nM insulin or with medium alone for 1 min. Insulin sensitizing effect of UCN was increased in dose (12B,12C) and time (12D) dependent manner. FIG. 12B was obtained by incubating cells with 2 nM insulin and variant dose of UCN (from 2 nM to 1 μM) for 1 min. FIG. 12C was obtained by incubating cells 100 nM UCN and variant dose of insulin for 1 min. Phosphorlyation of IR was assessed by western blotting with anti-pTyr antibodies. FIG. 13D was obtained by incubating cells with 100 nM UCN and/or 10 nM insulin for 0, 2, 10, 30, and 60 min, and then assessing the phosphorlyation of IR by western blotting with anti-pIR antibodies. Quantization of IR autophosphorylation was measured with image gauge software (Fuji film). The values are the mean±S.E. for three experiments. *,P<0.05

Cell Culture

hIRcB cells were maintained in DMEM, supplemented with 10% (v/v) FBS. The cells were grown at 37° C. in a humidified atmosphere of 5% CO₂ and 95% air.

Immunoprecipitation and Immunoblot

After treatment of ligands as indicated time and dose, the cells were washed with cold PBS and lysed with lysis buffer (50 mM HEPES pH 7.5, 150 mM NaCl, 1% Triton X-100, 1 mM EDTA, 10% glycerol) containing protease inhibitors (0.5 mM PMSF, 1 μg/ml leupeptin and 5 μg/ml aprotinin) and phosphatase inhibitors (30 mM NaF, 1 mM Na₃VO₄ and 30 mM Na₄O₇P₂). The cell lysates were incubated for 1 hr at 4° C. After centrifugation (14,000×g for 15 min), equal amounts of soluble extract were incubated, for 4 hrs, with 5 μg of anti-IR antibody and 30 μl of resin volume of immobilized protein A. For gel electrophoresis, the precipitates were dissolved in Laemmli sample buffer. The sample was separated by SDS-PAGE and transferred to a nitrocellulose membrane (Schleicher and Schuell, BA85). Blocking was performed with TTBS buffer (10 mM Tris/HCl, pH 7.5, 150 mM NaCl, and 0.05% Tween 20) containing 5% skimmed milk powder. The membrane was probed with primary for 3 hrs at room temperature. Subsequently the immunoblot was washed and incubated with horseradish peroxidase-linked secondary antibody for 1 hr at room temperature, rinsed four times in TTBS buffer, and developed with horseradish peroxidase-dependent chemiluminescence reagents (Amersham International, United Kingdom).

In this example, it was found that urocortin (UCN) and corticotropin releasing factor (CRF) potentate insulin-mediated IR phosphorylation compared with the other family, stress-related peptide (SRP) and stresscorpin (SCP) in IR over-expressed (hIRcB) cells (FIG. 12A). UCN and CRF alone have no effect on IR phosphorylation. Even though UCN and CRF potentate insulin-mediated IR phosphorylation, UCN is more potent for its high affinity to CRF receptor 1 (CRFR1) compared with CRF. The UCN-induced enhancement of insulin-mediated IR phosphorylation occurred in a dose-(FIG. 12B) and a time-dependent (FIG. 12D) manner. Furthermore, as shown in FIG. 12C, the effect of UCN was more potent in low concentration of insulin on IR phosphorylation. It is suggested that UCN specifically sensitizes insulin-mediated IR phosphorylation in hIRcB cells.

### EXAMPLE 9

The Effect of UCN on glucose uptake and IR phosphorylation in C2C12 myotubules.

UCN enhanced insulin-induced glucose uptake and phosphorylation of IR, IRS and Akt. Myotubules were incubated with 2 nM UCN and/or variant insulin dose (0-50 nM) for 1 min (Inserted immunoblot data). Phosphorylation of IRS and Akt was assessed by western blot analysis with anti-pIRS and anti-pAkt antibodies. Glucose uptake was determined as below.

Cell Culture

C2C12 cells were maintained in DMEM, supplemented with 10% (v/v) FBS. The cells were grown at 37° C. in a humidified atmosphere of 5% CO₂ and 95% air. For the differentiation of C2C12 cells, growing media was changed to DMEM, supplemented with 2% (v/v) ES and cultured for 7 days.

Glucose Uptake Assay

After differentiation, cells were washed and incubated during 3 hrs with 2 ml Krebs-Ringer phosphate (KRP) containing 130 mM NaCl, 5 mM KCl, 1.3 mM CaCl₂.2H₂O, 1.3 mM MgSO₄.7H₂O, and 10 mM Na₂HPO₄.7H₂O, pH 7.4. To determine the effect of UCN on glucose uptake, 10 min incubation in 1 ml of KRP at indicated conditions was carried out. The reaction was performed by adding a mixture of [¹⁴C] 2-DG (1 μCi/ml) and non-radioactive 2-DG (final concentration of 20 mM) for 10 min. The solution was removed by suction and the cells rapidly washed two times with ice-cold phosphate-buffered saline (PBS, containing 8 g of NaCl, 0.2 g of KCl, 1.15 g of Na₂HPO₄.12H₂0 and 0.2 g of KH₂PO₄ in 1 liter of H₂0). Cell-associated radioactivity was determined by lysing the cells in 1 N NaOH and followed by scintillation counting. Non-specific uptake was measured by incubating the cells with cytochalasin B (20 μM/ml). Non-specific uptake was subtracted from total uptake to obtain values for specific uptake.

Immunoprecipitation and immunoblot was performed according to the method of EXAMPLE 8.

It has been known that IR activation has pivotal role in the glucose uptake in muscle. UCN can potentiate the insulin-mediated IR activation and so it may regulate the glucose uptake. To confirm this, we investigated the glucose uptake and insulin-mediated signal in C2C12 myotubules. In the presence of UCN, insulin-induced phosphorylation of IR was enhanced and insulin-stimulated glucose uptake was also significantly increased (FIG. 13), but UCN alone was not. These results suggest that that UCN potentates insulin-stimulated glucose uptake in C2C12 myotubules, which may be induced through its role of sensitizer on insulin-mediated signal pathways.

### EXAMPLE 10

### Effects of UCN on Plasma Glucose Control in Normal and STZ-Mouse

UCN has been known as blood glucose enhancer by stimulating HPA axis. This in vivo function of UCN is opposite to our previous in vitro results, implicated in down-regulation of blood glucose level. Therefore, to discriminate the discrepancy between them, we injected diverse dose of UCN to mouse and checked the change of blood glucose level.

FIG. 14A showed that blood glucose was decreased in ICR mice by UCN. The inserted immunoblot data is about IR phosphorylation by UCN in skeletal muscle. Mice were injected (intravenously) with either vehicle (0.1% BSA in saline) or UCN (0.1-100 pM). Values are the mean±S.D. for four control and four UCN-treated mice. FIG. 14B showed that blood glucose was decreased in STZ mice by treatment of UCN. The inserted immunoblot data was about IR phosphorylation by treatment of UCN in skeletal muscle of STZ mouse. Mice were injected (intravenously) with vehicle (0.1% BSA in saline), UCN (0.1 pM) and/or insulin (1 nM). And plasma levels of glucose were measured during 45 min. Values are the mean±S.D. for six mice each group. *, P<0.05.

10-1: Preparation of Test Animals

For prepare the normal test animal, male Institute of Cancer Research (ICR) mice weighing 20-25 g, aged 8 weeks, were obtained from the Hyochang Science were housed four to cage in a temperature-and light-controlled room (20-22° C.; 12-hrs light, 12-hrs dark cycle; lights on at 07:00 hr) and were provided with regular diet chow and water ad libitum. The laboratory procedures used conformed to the guidelines of the Korea Government Guide for the Care of Use of Laboratory Animals. In the in vivo study, after fasting overnight, mice were injected to intravenous vein with 0.1% BSA saline or UCN, then plasma glucose was measured on a time by glucose analyzer (model AGM-2100, allmedicus Inc., anyang, Korea)

For preparing STZ-mouse, male Institute of Cancer Research (ICR) mice weighing 20-25 g, aged 8 weeks, were obtained from the Hyochang Science. STZ-induced diabetic mice were prepared by administering an intraperineal injection of STZ (Sigma Chemical, St. Louis, Mo.) (75 mg/kg) to male ICR mice after the animals were fasted for 1 day. And the next day had one more injection of STZ. Mice with plasma glucose concentrations≧280 mg/dl were considered to have type 1 diabetes. All tests were carried out 3 days after the injection of STZ.

10-2: Analysis of IR Signaling in Mouse Skeletal Tissue

After fasting overnight, mice were injected iv with agonist. After 15 min, mice were killed by soleus muscles were rapidly excised and were immediately frozen in liquid nitrogen. Lysates were prepared by homogenizing the tissues in lysis buffer (50 mM HEPES pH 7.5, 150 mM NaCl, 1% Triton X-100, 1 mM EDTA, 10% glycerol) containing protease inhibitors (0.5 mM PMSF, 1 μg/ml leupeptin and 5 μg/ml aprotinin) and phosphatase inhibitors (30 mM NaF, 1 mM Na₃VO₄ and 30 mM Na₄0₇P₂). Debris was removed by centrifugation at 14,000 rpm for 10 min at 4° C. Immunoprecipitations and Western blots were performed as previously described.

As expected, from 100 pM up, UCN alone enhanced blood glucose level, but, interestingly, 0.1 pM UCN alone down-regulated the blood glucose level. In contrast to in vitro system, basal insulin exists in blood and so we expected that UCN may have a glucose lowering effect with basal blood insulin in mouse and HPA axis may be not activated in sub-picomolar concentration. There is some possibility that UCN can modulate the insulin secretion and down-regulate blood glucose level independently with insulin. Therefore, we applied the insulin-deficient model system, streptozotocin (STZ)-treated mouse, to investigate the UCN function in insulin-mediated physiology.

As shown in FIG. 14B, UCN alone has no effect on the change of blood glucose level. It suggests that UCN could not independently role with insulin in the regulation of blood glucose level. However, when UCN was co-injected with inactive concentration of insulin, blood glucose level was significantly decreased in STZ-mice. These results were highly correlated with IR phosphorylation in mouse skeletal muscle. UCN significantly sensitized insulin-induced IR phosphorylation in mouse skeletal muscle. These results suggest that urocortin also has insulin-sensitizing effect in vivo.

What is claimed is:

1. A glucose uptake modulator which comprises a compound selected from the group consisting of lysophosphatidylcholine (LPC), lysophosphatidylserine(LPS), lysophosphatidic acid (LPA), and urocortin (UCN).

2. The glucose uptake modulator of claim 1, wherein the effect of LPC on glucose uptake are abrogated by the inhibitor of PKCδ, rottlerin, and expression of dominant negative PKCδ and are independent on PI3-kinase dependent signaling pathway.

3. The glucose uptake modulator of claim 1, wherein the lysophosphatidylcholine is Myristoyl LPC or palmytoyl LPC.

4. The glucose uptake modulator of claim 1, wherein urocortin acts as an insulin-sensitizing agent in combination of insulin.

5. A pharmaceutical composition which comprises at least a glucose uptake modulator selected from the group consisting of lysophosphatidylcholine, lysophosphatidylserine, lysophosphatidic acid, and urocortin.

6. The pharmaceutical composition of claim 5, wherein further comprises a pharmaceutically acceptable carrier, diluent or exipient.

7. The pharmaceutical composition of claim 5, wherein further comprises at least a compound selected from the group consisting of insulin secretion enhancers, biguanides, and α-glucosidase inhibitors.

8. The pharmaceutical composition of claim 5, wherein the glucose uptake modulator is used in combination with insulin.

9. A method for treating diabetes or diabetic complications in a mammal in need thereof, which comprises administering to said mammal an effecting amount of a glucose uptake modulator selected from the group consisting of lysophosphatidylcholine, lysophosphatidylserine, lysophosphatidic acid, or urocortin.

10. The method of claim 9, wherein the diabetes is insulin-dependent diabetes mellitus or noninsulin-dependent diabetes mellitus.

11. The method of claim 10, wherein the diabetic complication is obesity, hyperlipidemia, arteriosclerosis, hypertension or heart disease.

12. The method of claim 11 which farther comprises administering to said mammal an effecting amount of a glucose uptake modulator in combination of at least a compound selected from the group consisting of insulin secretion enhancers, biguanides, and α-glucosidase inhibitors.

13. The method of claim 9, wherein the urocortin is administered in combination with insulin.
